(19) United States
(12) Patent Application Publication
(10) Pub. No.: US 2016/0256102 A1
(43) Pub. Date: Sep. 8, 2016

(54) IDENTIFICATION OF RESPIRATION WAVEFORMS DURING CPR

(71) Applicant: ORIDION MEDICAL 1987 LTD., Jerusalem (IL)

(72) Inventors: Jacob Castiel, Herzeliya (IL); Keren Davidpur, Modi'in-Maccabim-Reut (IL); Michal RONEN, Givat-Brenner (IL)

(21) Appl. No.: 14/639,184

(22) Filed: Mar. 5, 2015

(51) Int. Cl. A61B 5/00; A61N 1/39; A61B 5/087; A61B 5/083; A61B 5/08

(52) U.S. Cl. CPC A61B 5/4836; A61B 5/0836; A61B 5/0816; A61B 5/725; A61B 5/7278; A61B 5/7264; A61B 5/087; A61B 5/7425; A61N 1/39; A61B 5/7405; A61B 2562/0247

(57) ABSTRACT

Device and method for identifying respiration waveforms during cardiopulmonary resuscitation (CPR), the method including obtaining CO₂ measurements from a subject undergoing CPR, processing the CO₂ measurements by applying a waveform detection algorithm thereto, wherein the waveform detection algorithm is configured to identify respiration waveforms and filtering out compression derived waveform-like signals; and identifying a respiration rate of the subject based on the identified respiration waveforms.

### TECHNICAL FIELD

The present disclosure generally relates to the field of breath monitoring and cardiopulmonary resuscitation (CPR).

### BACKGROUND

The main purpose of cardiopulmonary resuscitation (CPR) is to restore partial flow of oxygenated blood to the brain and heart, so as to delay tissue death and extend the brief window of opportunity for a successful resuscitation without permanent brain damage. The CPR involves a series of chest compressions to create artificial blood circulation and artificial ventilation to allow blood oxygenation and CO₂ clearance. CPR guidelines recommend keeping a defined relatively low level of respiration rate during CPR.

Defibrillation is a common treatment for life-threatening cardiac dysrhythmias, ventricular fibrillation and pulse less ventricular tachycardia. Defibrillation consists of delivering a therapeutic dose of electrical energy to the heart with a defibrillator device. This depolarizes a critical mass of the heart muscle, terminates the dysrhythmia and allows normal sinus rhythm to be reestablished. Defibrillators can be external, transvenous, or implanted. Some external units, known as automated external defibrillators (AEDs), automate the diagnosis of treatable rhythms, meaning that lay responders or bystanders are able to use them successfully with little or no training at all. Typically, the AEDs are configured to instruct CPR after each shock.

### SUMMARY

Aspects of the disclosure, in some embodiments thereof, relate to devices and methods for identifying respiration waveforms during CPR.

The CPR involves a series of chest compressions which bring about artificial blood circulation and artificial ventilation to allow blood oxygenation and CO₂ clearance. In order for CPR to be effective, the compression rate is recommended to be at least 100 compressions per minute and the respiration rate should be maintained relatively low namely 8-10 breaths per minute and no more than 15 breaths per minute.

Studies have shown that lay persons and even trained personnel tend to hyper ventilate the patient when providing CPR. Ventilation rates as high as about 40 breaths per minute are often provided. Interestingly even when advised to slow down ventilation, the average ventilation rate provided is still higher than 20 breaths per minute. During the decompression phase in CPR, a vacuum is created within the chest, drawing venous blood back to the heart. Frequent ventilations mean that less blood returns to the right heart between compressions, potentially reducing the effectiveness of CPR and thus contribute to the low survival rate of cardiac arrest.

In order to maintain adequate respiration rate (RR), a continuous indication of the RR is required. The RR may be calculated by identifying individual breath cycles and determining the time between subsequent breath cycles. However, accurate identification of respiration waveforms may be disrupted by the compression provided which generate a noise in the respiration signal.

Advantageously, the device and method for identifying respiration waveforms during CPR, disclosed herein, are configured to filter out alterations in the respiratory signal caused by compressions, thereby enabling accurate determination of the current respiration rate. Furthermore, the devise and method, disclosed herein may provide instructions as to the timing of ventilation required to obtain a desired respiration rate. This may enable a lay person to provide efficient CPR. Furthermore, even an experienced medical caregiver, such as a paramedic may utilize the method in order to provide optimal CPR while enabling him to accomplish additional tasks requiring his attention. Advantageously, method disclosed herein may further be applied in a closed loop of automated CPR and/or external defibrillator devices.

In addition, the method for identifying respiration waveforms during CPR, disclosed herein, may enable the identification of a respiration pattern responsive to the CPR. The identified respiration pattern may subsequently be used to adjust CPR parameters, such as but not limited to frequency of ventilation, depth of ventilation and the like. As the identified respiration pattern may be individual to each subject, the identification of the respiration pattern may enable the caregiver to provide personalized CPR.

As a further advantage, the identification of the respiration pattern responsive to the CPR may enable identification of underlying causes of a cardiac arrest. According to some embodiments, identification of the respiration pattern responsive to the CPR may enable to differentiate between ventricular fibrillation or pulseless ventricular tachycardia for which defibrillation typically is successful and asystole or pulseless electrical activity for which defibrillation typically is ineffective.

According to some embodiments, there is provided a method for identifying respiration waveforms during cardiopulmonary resuscitation (CPR) in a subject suffering from cardiac arrest, the method including: obtaining a plurality of CO₂ measurements from the subject undergoing CPR, processing the plurality of CO₂ measurements by applying a waveform detection algorithm thereto, and identifying a respiration rate of the subject based on the identified respiration waveforms. According to some embodiments, the measurements may include compression derived waveform-like signals. According to some embodiments, the waveform detection algorithm may be configured to identify respiration waveforms and filtering out compression derived waveform-like signals.

According to some embodiments, the waveform detection algorithm may be configured to identify respiration waveforms based on an identification of at least one waveform parameter. According to some embodiments, the at least one waveform parameter may include a shape and/or a scale factor.

According to some embodiments, applying the waveform detection algorithm may include applying differentials and/or heuristic rules to the plurality of CO₂ measurements.

According to some embodiments, the method may further include obtaining a plurality of flow and/or pressure sensor signals. According to some embodiments, identifying respiration waveforms in the plurality of CO₂ measurements may further include processing the plurality of flow or pressure sensor signals.

According to some embodiments, the method may further include obtaining a background characteristic of the subject. According to some embodiments, identifying respiration waveforms in the plurality of CO₂ measurements may further be based on the obtained background characteristic.

According to some embodiments, the method may further include obtaining a signal indicative of a compression. According to some embodiments, identifying respiration waveforms in the plurality of CO₂ measurements may further be based on the signal indicative of a compression.

According to some embodiments, the method may further include displaying the plurality of CO₂ measurements and/or the identified respiration waveforms.

According to some embodiments, the method may further include identifying a respiration pattern responsive to CPR based on an analysis of plurality of CO₂ measurements, the identified respiration waveforms and/or the compression derived waveform-like signals.

According to some embodiments, the method may further include adjusting at least one CPR parameter based on said identified respiration pattern. According to some embodiments, the adjustment of the at least one CPR parameter is automatic.

According to some embodiments, the at least one CPR parameter may be selected from frequency of chest compressions, depth of chest compressions, provision of breath, frequency of breath provision, target of breath provision, airway management or any combinations thereof.

According to some embodiments, there is provided processor configured to identify respiration waveforms during CPR, the processor including a control logic configured to: obtain a plurality of CO₂ measurements from a subject undergoing CPR, to process the plurality of CO₂ measurements by applying a waveform detection algorithm thereto, and to identify a respiration rate of the subject based on the identified respiration waveforms. According to some embodiments, the measurements may include compression derived waveform-like signals. According to some embodiments, the waveform detection algorithm may be configured to identify respiration waveforms and filtering out compression derived waveform-like signals.

According to some embodiments, the processor may further include (or be connected to) a display configured to display the plurality of CO₂ measurements and/or the identified respiration waveforms.

According to some embodiments, there is provided a medical device including a processor configured to: obtain a plurality of CO₂ measurements from a subject undergoing CPR, process the plurality of CO₂ measurements by applying a waveform detection algorithm thereto, and identify a respiration rate of the subject based on the identified respiration waveforms. According to some embodiments, the measurements may include compression derived waveform-like signals. According to some embodiments, the waveform detection algorithm may be configured to identify respiration waveforms and filtering out compression derived waveform-like signals.

According to some embodiments, the medical device may be an automated external defibrillator.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1 schematically shows a normal CO₂ waveform with sections A-F indicated;

FIG. 2 shows representative capnogram obtained during CPR, according to some embodiments;

FIG. 3A shows a representative capnogram obtained during CPR in a subject having a respiration pattern essentially unaffected by the CPR;

FIG. 3B shows a representative capnogram obtained during CPR in a subject having a respiration pattern mildly affected by the CPR;

FIG. 3C shows a representative capnogram obtained during CPR in a subject having a respiration pattern severely affected by the CPR;

FIG. 4 schematically shows a processor configured to identify respiration waveforms during CPR, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates generally to the field of breath monitoring during cardiopulmonary resuscitation (CPR).

There is provided, according to some embodiments, a method for identifying respiration waveforms during CPR. According to some embodiments the method includes obtaining a plurality of CO₂ measurements from a subject undergoing CPR; processing the plurality of CO₂ measurements by applying a waveform detection algorithm thereto; and identifying respiration waveforms in the plurality of CO₂ measurements based on the waveform detection algorithm applied on the plurality of CO₂ measurements.

As referred to herein, the terms “patient” and “subject” may interchangeably be used and may relate to a subject undergoing CPR. According to some embodiments, the subject may suffer from cardiac arrest.

As referred to herein, the term “waveform detection algorithm” may refer to an algorithm capable of identifying respiration waveforms in CO₂ measurements. According to some embodiments, the CO₂ measurements may be obtained from a CO₂ sensor. According to some embodiments, the CO₂ measurements may be obtained from a capnograph. According to some embodiments, the CO₂ measurements may be CO₂ concentration measurements. According to some embodiments, the CO₂ measurements may be partial pressure of CO₂ measurements. According to some embodiments CO₂ measurements may be volumetric CO₂ measurements.

According to some embodiments, the waveform detection algorithm may include differentials and/or heuristic rules. According to some embodiments, the waveform detection algorithm may use differentials to detect a regular shaped breath. According to some embodiments, the waveform detection algorithm may use heuristic rules to combine together respiration signals into a regular respiratory waveform.

As used herein, the term “respiration waveform” may refer to a CO₂ waveform obtained as a result of respiration and having a duration, shape and/or scale characteristic to respiration.

As used herein, the term “respiration signal” may refer to a signal indicative of a respiration waveform. Non-limiting examples of respiration signals include a predetermined time distance between waveforms, a decrease in the CO₂ level corresponding to a downwards slope of a respiration waveform, an increase in the CO₂ level corresponding to a upwards slope of a respiration waveform, a plateau in the CO₂ level indicative of a plateau of a respiration waveform, a CO₂ level corresponding to end tidal CO₂ (EtCO₂), or any other signal characteristic to a respiration waveforms. Each possibility is a separate embodiment.

According to some embodiments, applying a waveform detection algorithm may further include pre-processing a CO₂ measurement to reduce and/or filter out noise and/or artifacts. According to some embodiments, the waveform detection algorithm may enable the detection of abnormal events, such as but not limited to a sudden down-stroke in the CO₂ level, which may be result of a compression provided to the patient.

As used herein, the term “compression derived waveform-like signal” may refer to a waveform like signal, which is not originating from respiration. According to some embodiments, the compression derived waveform-like signals may originate from a compression provided to the subject during CPR. According to some embodiments, the compression derived waveform-like signal may differ from a respiration waveform in its duration, shape and/or scale. Each possibility is a separate embodiment. According to some embodiments, the compression derived waveform-like signal may be an “extra” waveform obtained in between two subsequent respiration waveforms. According to some embodiments, the compression derived waveform-like signal may be obtained during a respiration waveform, for example due to a sudden down-stroke in the CO₂ level in the midst of a respiration waveform, such that the respiration waveform will be split into two respiration-like waveforms.

According to some embodiments, the method may include identifying a respiration rate of the subject based on the identified respiration waveforms.

According to some embodiments, the waveform detection algorithm may be configured to identify respiration waveforms and/or compression derived waveform-like signals based on an identification of at least one waveform parameter. According to some embodiments, the at least one waveform parameter may include a shape and/or scale factors.

As used herein, the term “scale factor” may refer to waveform values and/or ratios characterizing the size of the waveform. Non-limiting examples of suitable scale factors include height, width, width at half-height, duty cycle, inhalation to exhalation ratio (I:E) or any other value or combination of values. Each possibility is a separate embodiment.

As used herein, the term “shape factor” may refer to waveform values and/or ratios characterizing the shape or pattern of the waveform. Non-limiting examples of suitable shape factors include slope of up-stroke, slope of down-stroke, slope of plateau, or any other value or combination of values. Each possibility is a separate embodiment.

According to some embodiments, the waveform detection algorithm may further be configured to identify respiration waveforms and/or compression derived waveform-like signals based on an integrated analysis of at least two waveform signals (whether derived from respiration, compression and/or noise and/or artifact). According to some embodiments, the integrated analysis may include an analysis of the distance between the at least two waveforms and/or a comparison of at least one waveform parameter identified in each of the at least two waveforms.

According to some embodiments, identifying respiration waveforms in the plurality of CO₂ measurements may include filtering out compression derived waveform-like signals. According to some embodiments, identifying respiration waveforms in the plurality of CO₂ measurements may include integrating and/or combining compression derived waveform-like signals into one or more respiration waveforms.

As used herein, the term “plurality” when referring to CO₂ measurements may refer to continuous measurements of CO₂ levels over time obtained from a CO₂ sensor, capnograph and/or flow or pressure sensor during the CPR.

As used herein, the term “at least one” may refer to 1, 2, 3, 4, 5, 10 or more. Each possibility is a separate embodiment. As used herein, the term “at least two” may refer to 2, 3, 4, 5, 10 or more. Each possibility is a separate embodiment.

According to some embodiments, the method may include obtaining a plurality of flow and/or pressure sensor signals from a breath flow sensor. According to some embodiments, identifying respiration waveforms among the plurality of CO₂ measurements may include processing the plurality of flow sensor signal. It is understood by one of ordinary skill in the art that the breath flow signals may be obtained and analyzed in addition to or as an alternative to obtaining and analyzing the CO₂ measurement. According to some embodiments, the method may further include identifying a subject's respiratory volume based on combined measurements of expired CO₂ and tidal volume.

According to some embodiments, the method may further include obtaining a signal indicative of a compression. According to some embodiments, the signal indicative of a compression may be obtained from a defibrillator, such as but not limited to an automated external defibrillator (AED). According to some embodiments, the identification of respiration waveforms, in the plurality of CO₂ measurements, may further be based on the signal indicative of a compression.

According to some embodiments, the method may further include obtaining patient background characteristics. According to some embodiments, the identification of respiration waveforms among the plurality of CO₂ measurements may further be based on the background characteristics. Non-limiting examples of suitable background characteristics may include gender, age (infant, child, adolescence, adult, elderly), weight (underweight, normal, over weight, obese), background disease (e.g. asthma, COPD and the like) or any other suitable background characteristic or combinations thereof. Each possibility is a separate embodiment. It is understood by one of ordinary skill in the art that the expected and/or desired respiration waveform is different for subjects with different background characteristics. For example, the respiration rate of an infant (and thus the scale factors of the respiration waveform) is different from that of an adult.

According to some embodiments, the method may include displaying the plurality of CO₂ measurements on a display. According to some embodiments, the method may include displaying the respiration waveforms differently from compression derived waveform-like signal. According to some embodiments, the method may include displaying the respiration waveforms identified in the plurality of CO₂ measurements. According to some embodiments, the method may include displaying the respiration waveforms as superimposed on the totality of waveforms and/or on the entire CO₂ measurement.

According to some embodiments, the method may include identifying a respiration pattern responsive to the CPR based on the identified respiration waveform and/or compression derived waveform-like signals and on the respiration rate. Each possibility is a separate embodiment.

As used herein, the term “respiration pattern” may refer to the rate, timing and/or consistency of respiration obtained in response to and/or during CPR. Each possibility is a separate embodiment. As further discussed hereinbelow, it was surprisingly found the respiration pattern during CPR differed tremendously among different subjects. Whereas the respiration pattern of some subjects is essentially unaffected by the CPR, the respiration pattern of some subjects is mildly and even severely affected. As used herein, the term “essentially unaffected respiration pattern” may refer to a respiration pattern in which the respiration waveform is immediately visible. According to some embodiments, an essentially unaffected respiration pattern may refer to a respiration patter with few and less pronounced compression derived waveform-like signals. According to some embodiments, an essentially unaffected respiration pattern may have less than 5 compression derived waveform-like signal per 2 minutes of CO₂ measurements. As used herein, the term “mildly affected respiration pattern” may refer to a respiration pattern in which the respiration waveform is somewhat disturbed due to the compression derived waveform-like signals. According to some embodiments, a mildly affected respiration pattern may have 5-10 compression derived waveform-like signal per 2 minutes of CO₂ measurements. As used herein, the term “severely affected respiration pattern” may refer to a respiration pattern in which the respiration waveform is profoundly disturbed by the compression derived waveform-like signals. According to some embodiments, a severely affected respiration pattern may have more than 10 compression derived waveform-like signal per 2 minutes of CO₂ measurements. According to some embodiments, a severely affected respiration pattern may refer to a respiration pattern affected to such an extent that no real pattern is visible.

According to some embodiments, the method may further include adjusting at least one CPR parameter based on the identified respiration waveforms, the calculated respiration rate and/or the identified respiration pattern. Each possibility is a separate embodiment. According to some embodiment, the adjustment of the at least one CPR parameter may be automatic. For example, when using an automated external defibrillator (AED), the method may function as a closed loop automatically adjusting the CPR parameter in response to the identified respiration waveforms and/or the respiration rate calculated therefrom.

According to some embodiments, the at least one CPR parameter may include frequency of chest compressions, depth of chest compressions, provision of breath, frequency of breath provision, target of breath provision (mouth and/or nose), airway management (e.g. head tilt) or any other parameter or combinations of parameters. Each possibility is a separate embodiment. For example, the identified respiration pattern may indicate that the frequency of chest compressions should be elevated/lowered (typically a frequency of 100 compressions per minute is recommended) or that the depth of compression is insufficient/or too high (typically a depths of 2 inch is recommended). Similarly, the identified respiration pattern may be indicative of whether provision of breath is recommended and whether adjustment of the frequency of breath provision is required.

According to some embodiments, the adjustment of the at least one CPR parameter may be further based on the background characteristics of a patient. Suitable patient characteristics have been described herein. As a non-limiting example, the desired depth of compression may differ among subjects of different age (e.g. infants vs. adults) and weight (e.g. normal vs. obese).

According to some embodiments, the method may further include identifying a degree of severity of the subject's respiratory status, based on the identified respiration pattern during CPR. For example, an irregular respiration pattern during CPR may be indicative of a more severe respiratory status than a regular respiration pattern during CPR.

According to some embodiments, the method may further include identifying an underlying cause of cardiac arrest based on the identified respiration pattern. According to some embodiments, identification of the respiration pattern responsive to CPR may enable to differentiate between ventricular fibrillation or pulseless ventricular tachycardia for which defibrillation typically is successful and asystole or pulseless electrical activity for which defibrillation typically is ineffective. According to some embodiments, the method may further include adjusting the at least one CPR parameter based on the identified underlying cause of the cardiac arrest.

According to some embodiments, there is provided a medical device comprising a processor configured to identify respiration during CPR. According to some embodiments, the medical device may include an automated external defibrillator (AED). According to some embodiments, the processor may be configured to obtain a plurality of CO₂ measurements from a subject undergoing CPR; and process the plurality of CO₂ measurements by applying a waveform detection algorithm. According to some embodiments, the waveform detection algorithm may be configured to identify respiration waveforms among the plurality of CO₂ measurements, as essentially described herein.

According to some embodiments, the processor may further be configured to calculate a respiration rate based on the identified respiration waveforms, as essentially described herein.

According to some embodiments, the processor may further be configured to identify a respiration pattern based on the identified respiration waveforms and on the respiration rate, as essentially described herein.

According to some embodiments, the processor may include a display configured to display the plurality of CO₂ measurements, the respiration waveforms and or the respiration pattern, as essentially described herein. According to some embodiments, the display may also display the respiration rate calculated from the identified respiration waveforms.

According to some embodiments, the processor may be configured to adjust at least one CPR parameter based on the identified respiration waveforms, the calculated respiration rate and/or the identified respiration pattern. Each possibility is a separate embodiment. According to some embodiment, the adjustment of the at least one CPR parameter may be automatic. For example, when using an automated external defibrillator (AED), the processor may operate in a closed loop automatically adjusting the CPR parameter in response to the identified respiration waveforms and/or on the respiration rate calculated therefrom, and/or the identified respiration pattern. Each possibility is a separate embodiment.

According to some embodiments, the processor may further be configured to provide instructions concerning at least one parameter of the CPR, based on the identified respiration waveforms, the respiration rate and/or the respiration pattern. As a non-limiting example, the processor may provide an instruction to reduce the depth of compression or to reduce the frequency of breath provision. According to some embodiments, the instruction may be a written instruction displayed on the display. According to some embodiment, the instruction may be a vocal instruction.

According to some embodiments, the processor may be configured to trigger an alarm if the identified respiration waveforms, the respiration rate and/or the respiration pattern deviate from normal by a predetermined threshold value.

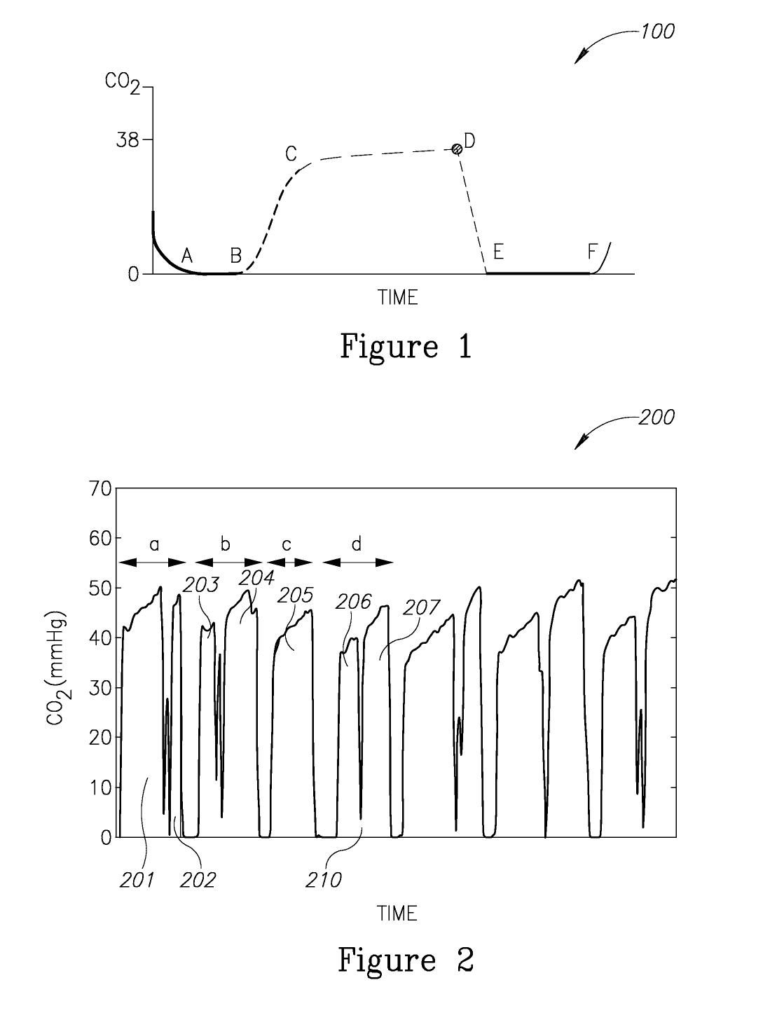

Reference is now made to FIG. 1, which shows CO₂ measurements including a “normal” (textbook) CO₂ waveform 100. CO₂ waveform 100 represents the varying CO₂ level throughout the respiratory cycle. Points A, B, C, D, E and F are depicted on waveform 100. Section (A-B) represents the end of inspiration (Phase I), where the CO₂ level is zero. Point B represents the beginning of exhalation. The sharp upstroke (B-C) represents the exhalation (Phase II). Follows is a gradual rise (C-D) (Phase III), a plateau having a peak just before (D) which represents the end of exhalation. The sharp down-stroke back to zero (D-E) represents the inspiration (Phase IV) and is followed by a clean inspiration period (section E-F).

Reference is now made to FIG. 2 which shows a representative capnogram 200 obtained during CPR, according to some embodiments. Capnogram 200 includes a plurality of CO₂ waveforms, such as waveforms 201-207. Waveform 205 represents a normal waveform which can be directly interpreted as a respiration waveform, such as respiration waveform c. However, waveforms 201-204 and 206-207 are waveforms which do not represent respiration waveforms (also referred to herein as compression derived waveform-like signals). It is understood to one of ordinary skill in the art that without further processing, waveforms 201-204 and 206-207 may be interpreted as respiration waveforms and may therefore indicate an exaggeratedly high respiration rate. Similarly, the maximum concentration of CO₂ of waveforms 201, 203 and 206 may be falsely interpreted as an abnormally low EtCO₂ level. Advantageously, the method disclosed herein enables the determination of respiration waveforms from the waveforms in the plurality of CO₂ measurements, such as determining respiration waveforms a-d from waveforms 201-207. The determination of respiration waveforms a-d may be based on an analysis of the shape and scale of the totality of waveforms in the plurality of CO₂ measurements, the distance between the waveforms as well as other waveform parameters, as essentially described herein. In short, the analysis may include applying a waveform detection algorithm on the plurality of CO₂ measurements. The analysis of the plurality of CO₂ measurements may further enable to identify abnormal events in a waveform, such as a sudden down-stroke in the waveform (i.e. down-stroke 210) representing a sudden drastic reduction in the CO₂ level which may be a result of a compression provided to the patient. Alternatively, when a defibrillator is used, the compression may be provided as an input signal to the algorithm. It is understood to one of ordinary skill in the art that the identification of abnormal events (i.e. compression related changes in the CO₂ signal) may assist in the identification of true respiration waveforms. It is further understood that once respiration waveforms (such as respiration waveforms a-d) have been identified a true respiration rate may readily be calculated.

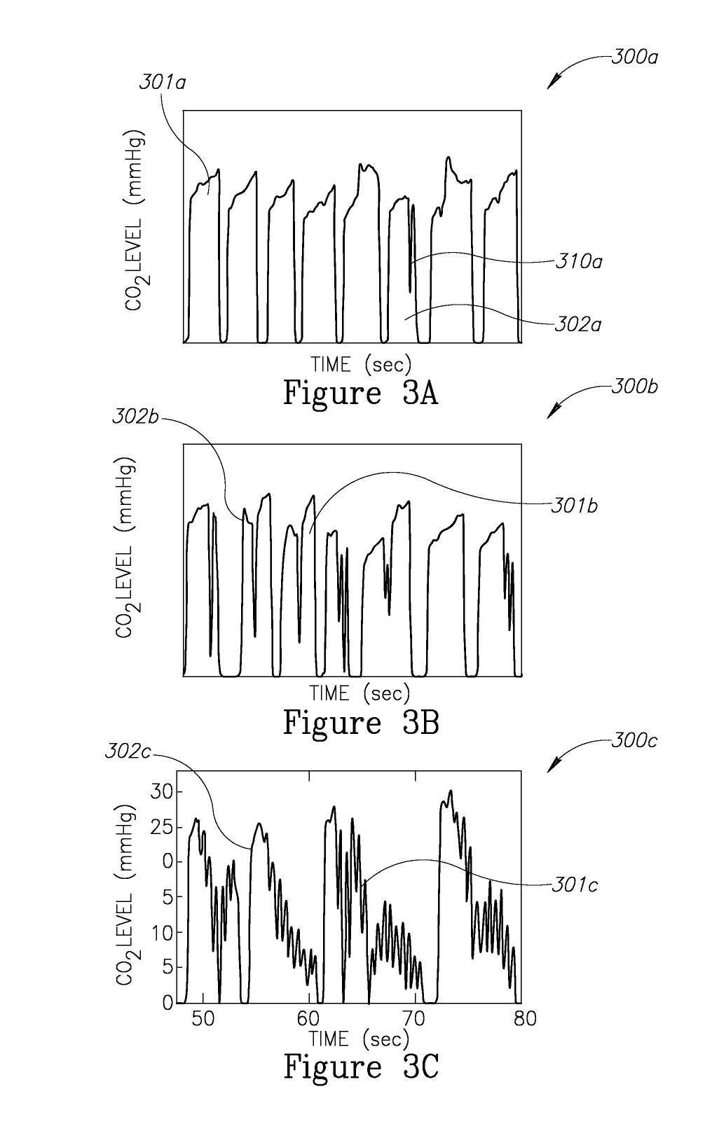

Reference is now made to FIG. 3A which shows a representative capnogram 300a obtained during CPR in a subject with a respiration pattern largely unaffected by the CPR, according to some embodiments. Capnogram 300a includes a plurality of CO₂ measurements such as waveforms 301a that can largely be directly interpreted as respiration waveform, without much processing. Although abnormal changes in the CO₂ measurements can be identified (such as down-stroke 310a in waveform 302a), the reduction is relatively minor and a normal respiration pattern is largely preserved.

Reference is now made to FIG. 3B which shows a representative capnogram 300b obtained during CPR in a subject with a respiration pattern mildly affected by the CPR, according to some embodiments. Capnogram 300b includes a plurality of CO₂ signals some of which are not true respiration waveforms, such as for example waveforms 301b and 302b. Processing, such as the processing disclosed herein, enables the identification of respiration waveforms from waveforms 301b-302b.

Reference is now made to FIG. 3C which shows a representative capnogram 300c obtained during CPR in a subject with a respiration pattern severely affected by the CPR, according to some embodiments. Capnogram 300c includes a plurality of CO₂ signals, such as for example waveforms 301c and 302c, which only slightly resemble respiration waveforms. Processing, such as the processing disclosed herein, is required in order to extrapolate respiration waveforms from the obtained CO₂ signals.

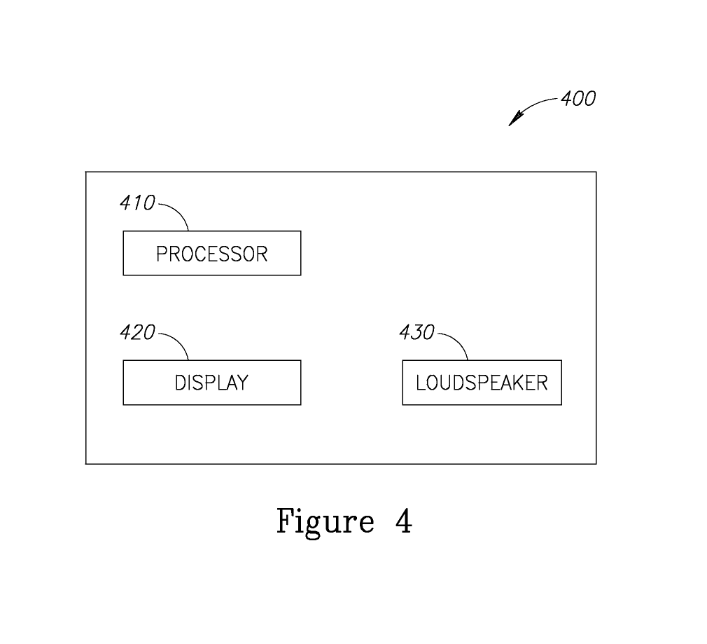

Reference is now made to FIG. 4, which schematically shows a medical device 400, such as but not limited to an automated external defibrillator (AED) configured to identify respiration waveforms during CPR, according to some embodiments. Medical device 400 includes a processor 410 configured to execute the method as essentially described herein. In short, medical device 400 includes a processor 410 configured to obtain a plurality of CO₂ measurements from a subject undergoing CPR. Processor 410 is further configured to process the plurality of CO₂ measurements by applying a waveform detection algorithm, thereby identifying respiration waveforms among the plurality of CO₂ measurements. Processor 410 is further configured to calculate a respiration rate based on the identified respiration waveforms. Optionally, processor 410 may further be configured to identify a respiration pattern based on the identified respiration waveform. Processor 410 is further configured to adjust at least one CPR parameter based on the identified respiration waveforms, the calculated respiration rate and/or the identified respiration pattern. Optionally, the adjustment of the at least one CPR parameter may be automatic. For example, when part of an AED, processor 410 may operate in a closed loop automatically adjusting the CPR parameters of the AED in response to the identified respiration waveforms, the respiration rate calculated therefrom, and/or the identified respiration pattern. Medical device 400 further includes a display 420 configured to display the plurality of CO₂ measurements and/or the identified respiration waveforms. According to some embodiments, display 420 may also display the respiration rate calculated from the identified respiration waveforms. Optionally, medical device 400 may also include a loudspeaker 430. Loudspeaker 430 is configured to provide vocal instructions concerning at least one parameter of the CPR, based on the identified respiration waveforms, the respiration rate and/or the respiration pattern. Optionally, loudspeaker 430 may be configured to vocally inform the CPR provider of the respiration rate and/or the respiration pattern of the subject.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A method for identifying respiration waveforms during cardiopulmonary resuscitation (CPR) in a subject suffering from cardiac arrest, the method comprising:
obtaining a plurality of CO₂ measurements from the subject undergoing CPR, said measurements comprising compression derived waveform-like signals;
processing the plurality of CO₂ measurements by applying a waveform detection algorithm thereto, wherein the waveform detection algorithm is configured to identify respiration waveforms and filtering out compression derived waveform-like signals; and
identifying a respiration rate of the subject based on the identified respiration waveforms.

2. The method of claim 1, wherein the waveform detection algorithm is configured to identify respiration waveforms based on an identification of at least one waveform parameter.

3. The method of claim 2, wherein the at least one waveform parameter comprise a shape and/or a scale factor.

4. The method of claim 1, wherein applying the waveform detection algorithm comprises applying differentials and/or heuristic rules to the plurality of CO₂ measurements.

5. The method of claim 1, further comprising obtaining a plurality of flow sensor signals and/or pressure sensor signals.

6. The method of claim 5, wherein identifying respiration waveforms in the plurality of CO₂ measurements further comprises processing the plurality of flow sensor signals and/or pressure sensor signals.

7. The method of claim 1, further comprising obtaining a background characteristic of the subject.

8. The method of claim 7, wherein identifying respiration waveforms in the plurality of CO₂ measurements is further based on the obtained background characteristic.

9. The method of claim 1, further comprising obtaining a signal indicative of a compression.

10. The method of claim 9, wherein identifying respiration waveforms in the plurality of CO₂ measurements is further based on the signal indicative of a compression.

11. The method of claim 1, further comprising displaying the plurality of CO₂ measurements and/or the identified respiration waveforms.

12. The method of claim 1, further comprising identifying a respiration pattern responsive to CPR based on an analysis of plurality of CO₂ measurements, the identified respiration waveforms and/or the compression derived waveform-like signals.

13. The method of claim 12, further comprising adjusting at least one CPR parameter based on said identified respiration pattern.

14. The method of claim 13, wherein the at least one CPR parameter is selected from frequency of chest compressions, depth of chest compressions, provision of breath, frequency of breath provision, target of breath provision, airway management or any combinations thereof.

15. The method of claim 13, wherein the adjustment of the at least one CPR parameter is automatic.

16. A processor configured to identify respiration waveforms during CPR, the processor comprising a control logic configured to:
obtain a plurality of CO₂ measurements from a subject undergoing CPR, said measurements comprising compression derived waveform-like signals; and
process the plurality of CO₂ measurements by applying a waveform detection algorithm thereto, wherein said waveform detection algorithm is configured to identify respiration waveforms and filtering out compression derived waveform-like signals; and
identify a respiration rate of the subject based on the identified respiration waveforms.

17. The processor of claim 16, further comprising a display configured to display the plurality of CO₂ measurements and/or the identified respiration waveforms.

18. A medical device comprising a processor configured to:
obtain a plurality of CO₂ measurements from a subject undergoing CPR, said measurements comprising compression derived waveform-like signals; and
process the plurality of CO₂ measurements by applying a waveform detection algorithm thereto, wherein said waveform detection algorithm is configured to identify respiration waveforms and filtering out compression derived waveform-like signals; and
identify a respiration rate of the subject based on the identified respiration waveforms.

19. The medical device of claim 18 being an automated external defibrillator.
